(19) United States
(12) Patent Application Publication
(10) Pub. No.: US 2003/0195368 A1
(43) Pub. Date: Oct. 16, 2003

(54) Bisphosphite compounds, the metal complexes thereof and the use of said compounds and complexes in olefin hydroformylation

(76) Inventors: Dirk Rottger, Recklinghausen (DE); Renat Kadyrov, Frankfurt (DE); Armin Borner, Rostock (DE); Detlef Selent, Berlin (DE); Dieter Hess, Marl (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/311,322

(22) PCT Filed: Jun. 20, 2001
(86) PCT No.: PCT/EP01/06968

(30) Foreign Application Priority Data
Jun. 28, 2000 (DE) 100 31 493.7

(51) Int. Cl. C07F 9/02; C07F 15/00; C07C 45/49; C07C 29/15

(52) U.S. Cl. 556/13; 558/73; 568/451; 568/909

(57) ABSTRACT

The invention relates to the bisphosphites of the general formula(I), wherein R1, R2, R3, $4 R5 R6 represent H, an aliphatic, alicyclic, aliphatic-alicyclic, heterocyclic, aliphatic-heterocyclic, aromatic, aliphatic-aromatic hydrocarbon group with 1 to 50 carbon atoms, F, Cl, Br, I, —OR7, —COR7, —CO2R7, —CO2M, —SR7, —SO2R7, —SOR7, —SO3R7, —SO3M, —SO2NR7R8, NR7R8, N═CR7R8, NH2, wherein R1 to R6 are the same or different and can be covalently linked with each other, R7, R8 represent H, a substituted or unsubstituted, aliphatic or aromatic hydrocarbon group with 1 to 25 carbon atoms, which are the same or different, M represents an alkali metal, alkaline earth metal, ammonium, phosphonium ion; Q represents a bivalent, aliphatic alicyclic, aliphatic-alicyclic, heterocyclic, aromatic, aliphatic-aromatic hydrocarbon group with 1 to 50 carbons atoms, W, X represent aliphatic, alicyclic, aliphatic-alicyclic, heterocyclic, aliphatic-heterocyclic, aromatic, aliphatic-aromatic hydrocarbon groups with 1 to 50 carbon atoms, which are the same or different or which are covalently linked each other. The invention further relates to the metal complexes of said bisphosphites and to the use thereof in hydroformylation reactions.

The present invention relates to bisphosphites and their metal complexes and to the preparation and use of the bisphosphites as ligands in catalytic reactions.

The reactions between olefin compounds, carbon monoxide and hydrogen in the presence of a catalyst to form aldehydes having one more carbon atom is known as hydroformylation (oxo process). As catalysts for these reactions, use is frequently made of compounds of transition metals of group VIII of the Periodic Table of the Elements, in particular rhodium and cobalt compounds. Hydroformylation using rhodium compounds generally offers the advantage of higher selectivity compared to cobalt catalysis and is thus usually more economical. Rhodium-catalyzed hydroformylation is usually carried out using complexes comprising rhodium and preferably trivalent phosphorus compounds as ligands. Known ligands are, for example, compounds from the classes of phosphines, phosphites and phosphonites. A good overview of the state of the art of hydroformylation of olefins may be found in B. CORNILS, W. A. HERRMANN, “Applied Homogeneous Catalysis with Organometallic Compounds”, Vol. 1 & 2, VCH, Weinheim, N.Y., 1996.

Each catalyst system (cobalt or rhodium) has its specific advantages. For this reason, different catalyst systems are used depending on the starting material and the target product, as the following examples show. If rhodium and triphenylphosphine are employed, α-olefins can be hydroformylated at lower pressures. As phosphorus-containing ligand, use is generally made of an excess of triphenylphosphine; a high ligand/rhodium ratio is necessary to increase the selectivity of the reaction to the commercially desired n-aldehyde product.

The patents U.S. Pat. No. 4,694,109 and U.S. Pat. No. 4,879,416 describe bisphosphine ligands and their use in the hydroformylation of olefins at low synthesis gas pressures. Particularly in the hydroformylation of propene, high activities and high n/I selectivities are achieved using ligands of this type. WO 95/30680 discloses bidentate phosphine ligands and their use in catalysis, including their use in hydroformylation reactions. Ferrocene-bridged bisphosphines are described, for example, in the patents U.S. Pat. No. 4,169,861, U.S. Pat. No. 4,201,714 and U.S. Pat. No. 4,193,943 as ligands for hydroformylations.

The disadvantage of bidentate phosphine ligands is the relatively high cost of preparing them. It is therefore often not economically viable to use such systems in industrial processes.

Rhodium-monophosphite complexes are suitable catalysts for the hydroformylation of branched olefins having internal double bonds, but the selectivity to compounds containing terminal aldehyde groups is low. EP 0 155 508 discloses the use of bisarylene-substituted monophosphites in the rhodium-catalyzed hydroformylation of sterically hindered olefins, e.g. isobutene.

Rhodium-bisphosphite complexes catalyze the hydroformylation of linear olefins having terminal and internal double bonds to give predominantly terminally hydroformylated products, while branched olefins having internal double bonds react to only a small extent. On coordination to a transition metal center, these phosphites give catalysts of increased activity, but the operating life of these catalyst systems is unsatisfactory, partly because of the hydrolysis sensitivity of the phosphite ligands. Considerable improvements were able to be achieved by the use of substituted bisaryl diols as starting materials for the phosphite ligands, as described in EP 0 214 622 or EP 0 472 071.

According to the literature, the rhodium complexes of these ligands are extremely active hydroformylation catalysts for α-olefins. The patents U.S. Pat. No. 4,668,651, U.S. Pat. No. 4,748,261 and U.S. Pat. No. 4,885,401 describe polyphosphite ligands by means of which α-olefins and also 2-butene can be converted highly selectively into the products having terminal aldehyde groups. Bidentate ligands of this type have also been used for the hydroformylation of butadiene (U.S. Pat. No. 5,312,996).

Although the bisphosphites mentioned are very good complexing ligands for rhodium hydroformylation catalysts, it is desirable to achieve a further improvement in their effectiveness and hydrolysis resistance.

It has been found that bisphosphites having the structure I

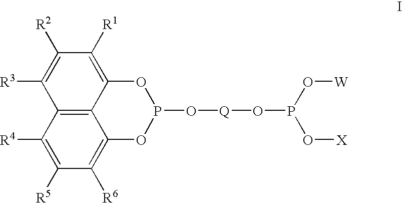

can be prepared in a simple manner and are suitable as ligands in metal-catalyzed reactions.

The present invention accordingly provides bisphosphites of the formula I

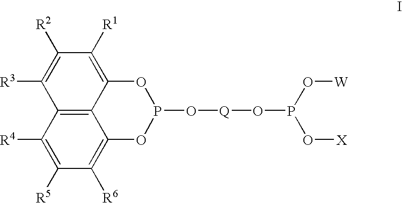

where

R¹, R², R³, R⁴, R⁵, R⁶=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR⁷, —COR⁷, —CO₂R⁷, —CO₂M, —SR⁷, —SO₂R⁷, —SOR⁷, —SO₃R⁷, —SO₃M, —SO₂NR⁷R⁸, NR⁷R⁸, N═CR⁷R⁸, NH₂, where R¹ to R⁶ may be identical or different and may be covalently linked to one another,

R⁷, R⁸=H or a substituted or unsubstituted, aliphatic or aromatic hydrocarbon radical having from 1 to 25 carbon atoms, where R⁷ and R⁸ may be identical or different,

M=an alkali metal ion, alkaline earth metal ion, ammonium ion or phosphonium ion,

Q=a divalent aliphatic, alicyclic, aliphatic-alicyclic, heterocyclic, aliphatic-heterocyclic, aromatic or aliphatic-aromatic hydrocarbon radical having from 1 to 50 carbon atoms,

W, X=aliphatic, alicyclic, aliphatic-alicyclic, heterocyclic, aliphatic-heterocyclic, aromatic or aliphatic-aromatic hydrocarbon radicals having from 1 to 50 carbon atoms, which may be identical or different or may be covalently linked to one another.

Specific embodiments of the bisphosphites of the invention are bisphosphites of the formulae II and III

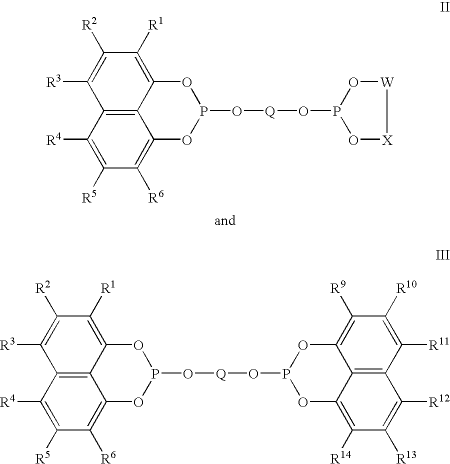

where

W and X are aliphatic, alicyclic, aliphatic-alicyclic, heterocyclic, aliphatic-heterocyclic, aromatic or aliphatic-aromatic hydrocarbon radicals having from 1 to 50 carbon atoms, where X and W may be identical or different or may be covalently linked to one another,

and R¹, R², R³, R⁴, R⁵, R⁶, R⁷, R⁸ and Q are as defined above.

R⁹, R¹⁰, R¹¹, R¹², R¹³, R¹⁴ are each H or an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR¹⁵, —COR¹⁵, —CO₂R¹⁵, —CO₂M, —SR¹⁵, —SO₂R ¹⁵, —SOR¹⁵, —SO₃R¹⁵, —SO₃M, —SO₂NR¹⁵R¹⁶, NR¹⁵R¹⁶, N═CR¹⁵R¹⁶, NH₂, where R⁹ to R¹⁴ are identical or different and may be covalently linked to one another.

M is an alkali metal ion, alkaline earth metal ion, ammonium ion or phosphonium ion.

R¹⁵ and R¹⁶ may be identical or different and are each H or a substituted or unsubstituted, aliphatic or aromatic hydrocarbon radical having from 1 to 25 carbon atoms.

Examples of Q are bivalent hydrocarbon radicals which may be aliphatic, alicyclic, aliphatic-alicyclic, heterocyclic, aliphatic-heterocyclic, aromatic or aliphatic-aromatic. Any ring systems present may in turn be substituted by the abovementioned hydrocarbon radicals. In open-chain structural elements, one or more of the methylene groups may be replaced by oxygen and/or sulfur and/or NR⁷ and/or NH and/or one or more CH groups may be replaced by nitrogen.

Q are preferably bivalent radicals containing aromatic groups. Q can be, for example, a phenylene radical, a naphthylene radical, a divalent bisarylene radical or a bivalent radical of a diphenyl ether. Furthermore, Q can have the structure —Ar—Z—Ar—. Here, Ar is a monocyclic or polycyclic bivalent aromatic radical. Z is either a direct bond or a substituted or unsubstituted methylene group —CR⁷R⁸—, where R⁷ and R⁸ are each hydrogen or an aliphatic and/or aromatic radical which has from 1 to 25 carbon atoms and may also contain heteroatoms. Furthermore, the radicals R⁷ and R⁸ may be joined to form one or more rings, i.e. have a covalent bond.

Among the bisphosphites of the formulae I, II and III, particular preference is given to those in which the radical Q is a hydrocarbon radical (bisarylene radical) of the formula IV

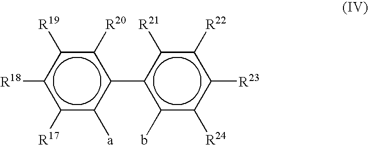

where

R¹⁷, R¹⁸, R¹⁹, R²⁰, R²¹, R²², R²³, R²⁴=H, aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR²⁵, —COR²⁵, —CO₂R²⁵, —CO₂M, —SR²⁵, —SO₂R²⁵, —SOR²⁵, —SO₃R²⁵, —SO₃M, —SO₂NR²⁵R²⁶, NR²⁵R²⁶, N═CR²⁵R²⁶, NH₂, where R¹⁷ to R²⁴ are identical or different and may be covalently linked to one another,

R²⁵, R²⁶=H or a substituted or unsubstituted, aliphatic or aromatic hydrocarbon radical having from 1 to 25 carbon atoms,

M=an alkali metal ion, alkaline earth metal ion, ammonium ion or phosphonium ion,

where the positions a and b represent linkage points of this substituent in the structural element O—Q—O in the compounds of the formulae I, II and III.

Examples of W and X are hydrocarbon radicals which may be aliphatic, alicyclic, aliphatic-alicyclic, heterocyclic, aliphatic-heterocyclic, aromatic or aliphatic-aromatic. Ring systems present in the radicals may in turn be substituted by the hydrocarbon radicals mentioned. In open-chain structural elements, one or more methylene groups may be replaced by oxygen and/or sulfur and/or NR⁷ and/or NH and/or one or more CH groups may be replaced by nitrogen.

The present invention also provides bisphosphite-metal complexes comprising a metal of transition group 4, 5, 6, 7 or 8 of the Periodic Table of the Elements and one or more bisphosphites of the formulae I, II and III. The substituents (R¹-R²⁴, Q, X, W) of these bisphosphites have the abovementioned meanings.

Representative examples of ligands of the general formulae I, II, III according to the present invention are shown below, without the scope of the present invention being restricted thereby.

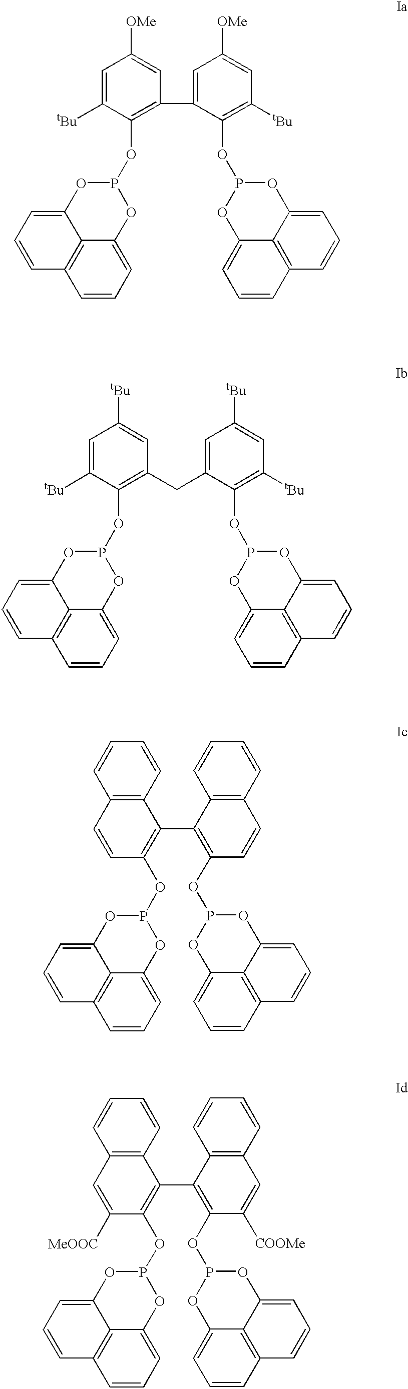

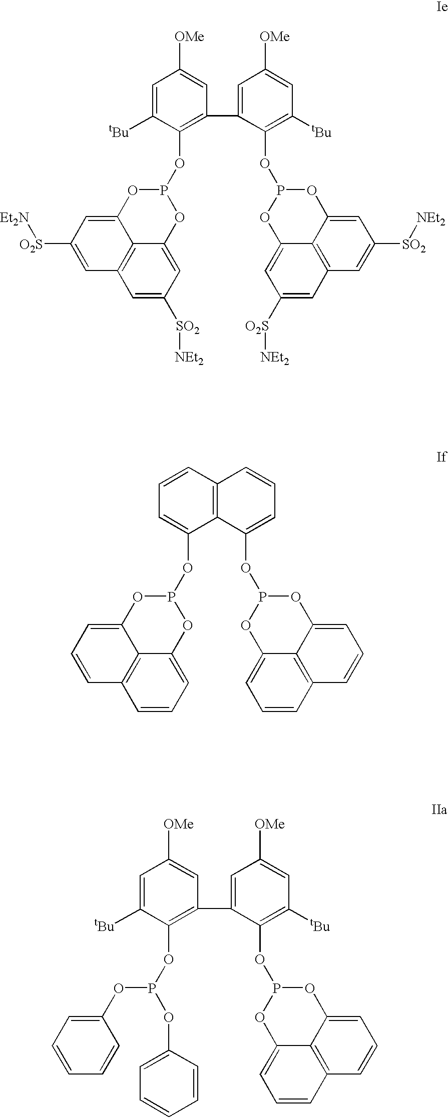

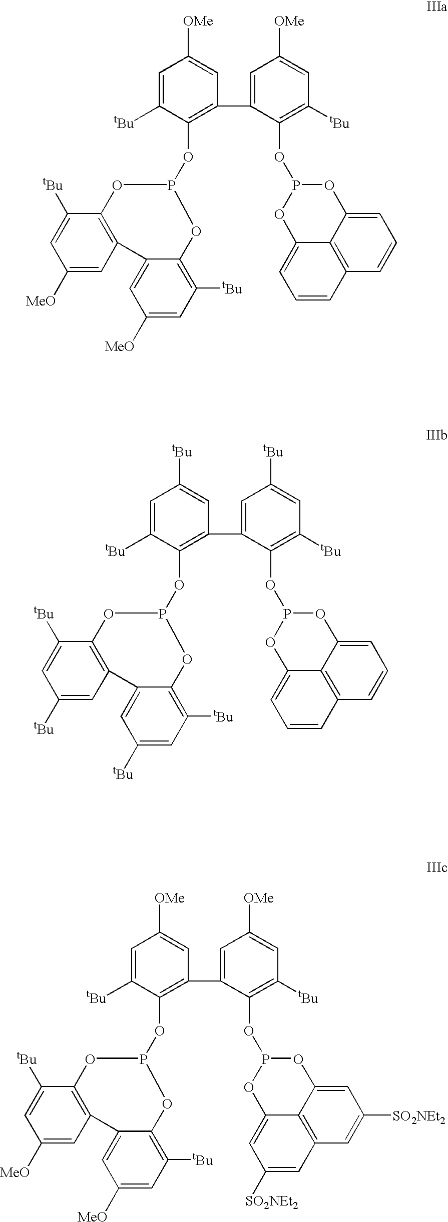

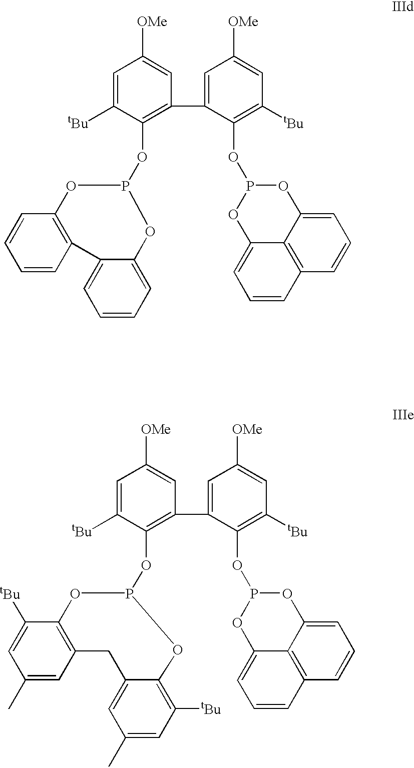

The bisphosphites of the invention can be prepared by reactions of phosphorus halides with alcohols, in which halogen atoms are replaced by alkoxide groups:

a) A phosphorus trihalide, preferably phosphorus trichloride, is reacted with a diol or two molar equivalents of alcohol to form a monohalophosphite (intermediate A);

b) the intermediate A is reacted with a diol (HO—Q—OH) to give a hydroxyl-substituted phosphite (intermediate B);

c) a phosphorus trihalide, preferably phosphorus trichloride, is reacted with substituted or unsubstituted 1,8-dihydroxynaphthalene to form a monohalophosphite (C);

d) the intermediate B is reacted with C to give the desired bisphosphite.

Symmetrical bisphosphites of the formula III can be obtained in an even simpler fashion by reaction of the component C with a diol.

Since the diols used and their downstream products are frequently solid, the reactions are generally carried out in solvents. Solvents used are aprotic solvents which react neither with the diols nor with the phosphorus compounds. Examples of suitable solvents are tetrahydrofuran, diethyl ether and aromatic hydrocarbons carbons such as toluene.

The reaction of phosphorus halides with alcohols forms a hydrogen halide which is bound by added bases. For example, tertiary amines such as triethylamine are used. It is also possible to convert the alcohols into metal alkoxides prior to the reaction, for example by reacting them with sodium hydride or butyllithium.

Apart from this synthetic route, there are other suitable methods of preparing the bisphosphite ligands of the invention. They include, for example, the use of tris(dialkylamino) phosphines (as alternatives to phosphorus trichloride).

The 1,8-dihydroxynaphthalene building block used in the syntheses can likewise be obtained in a variety of ways. Thus, for example, 1,8-dihydroxynaphthalene itself can be obtained from 1,8-naphthalene sulfone by reaction with potassium hydroxide (L. Ann. Chem. 1888, 247, 356). In addition, derivatives of chromotropic acid (4, 5-dihydroxy-2,7-naphthalenedisulfonic acid) are possible starting materials.

The novel bisphosphites of the formulae I, II and III are suitable building blocks for the preparation of complexes with metals of transition groups 4, 5, 6, 7 and 8 of the Periodic Table of the Elements. These complexes, particularly those of metals of transition group 8, can be used as catalysts for carbonylation reactions or hydroformylation reactions, e.g. for the hydroformylation of C2-C25-olefins. The ligands have a high hydrolysis stability. Particularly when using rhodium as catalyst metal, they display high catalytic activities in hydroformylation reactions. Owing to their high molecular weight, the bisphosphites of the invention have a low volatility. They can therefore easily be separated off from the more volatile reaction products. They are sufficiently soluble in customary organic solvents.

The invention also provides for the use of the bisphosphites or the bisphosphite-metal complexes in processes for the hydroformylation of olefins, preferably olefins having from 2 to 25 carbon atoms, to give the corresponding aldehydes.

To prepare the catalytically active metal complexes from the bisphosphites of the invention, preference is given to using the metals rhodium, cobalt, platinum and ruthenium. The active catalyst is formed from the ligands of the invention and the metal under the reaction conditions. The ligands of the invention can be added in free form to the reaction mixture. It is also possible to use a transition metal complex containing the abovementioned bisphosphite ligands as precursor for the actual catalytically active complex. The hydroformylation process can be carried out using a stoichiometric amount or any excess of free bisphosphite ligands.

It is also possible for mixtures of various ligands, both the bisphosphites of the invention and other suitable phosphorus-containing ligands, to be present as free ligand components.

As additional ligands present in the reaction mixture, it is possible to use phosphines, phosphites, phosphonites or phosphinites.

Examples of such ligands are:

phosphines: triphenylphosphine, tris(p-tolyl)phosphine, tris(m-tolyl)phosphine, tris(o-tolyl)phosphine, tris(p-methoxyphenyl)phosphine, tris(p-dimethylaminophenyl)phosphine, tricyclohexylphosphine, tricyclopentylphosphine, triethylphosphine, tri(1-naphthyl)phosphine, tribenzylphosphine, tri-n-butylphosphine, tri-t-butylphosphine;

phosphites: trimethyl phosphite, triethyl phosphite, tri-n-propyl phosphite, tri-i-propyl phosphite, tri-n-butyl phosphite, tri-i-butyl phosphite, tri-t-butyl phosphite, tris(2-ethylhexyl) phosphite, triphenyl phosphite, tris(2,4-di-t-butylphenyl) phosphite, tris(2-t-butyl-4-methoxyphenyl)phosphite, tris(2-t-butyl-4-methoxyphenyl)phosphite, tris(p-cresyl) phosphite, also sterically hindered phosphite ligands as are described, inter alia, in EP 155 508, U.S. Pat. No. 4,668,651, U.S. Pat. No. 4,748,261, U.S. Pat. No. 4,769,498, U.S. Pat. No. 4,774,361, U.S. Pat. No. 4,835,299, U.S. Pat. No. 4,885,401, U.S. Pat. No. 5,059,710, U.S. Pat. No. 5,113,022, U.S. Pat. No. 5,179,055, U.S. Pat. No. 5,260,491, U.S. Pat. No. 5,264,616, U.S. Pat. No. 5,288,918, U.S. Pat. No. 5,360,938, EP 472 071, EP 518 241 and WO 97/20795;

phosphonites: methyldiethoxyphosphine, phenyldimethoxyphosphine, phenyldiphenoxyphosphine, 6-phenoxy-6H-dibenz[c,e][1,2]oxaphosphorin and its derivatives in which all or some of the hydrogen atoms are replaced by alkyl or aryl radicals or halogen atoms, and ligands described in WO 98 43935, UP 09-268152 and DE 198 10 794 and in the German patent applications DE 199 54 721 and DE 199 54 510;

phosphinite ligands: as described, inter alia, in U.S. Pat. No. 5,710,344, WO 95 06627, U.S. Pat. No. 5,360,938 or JP 07082281, for example diphenyl(phenoxy)phosphine and its derivatives in which all or some of the hydrogen atoms are replaced by alkyl or aryl radicals or halogen atoms, diphenyl(methoxy)phosphine, diphenyl(ethoxy)phosphine, etc.

Use is generally made of from 1 to 500 mol, preferably from 1 to 200 mol, more preferably from 3 to 50 mol, of the ligand of the invention per mol of Group VIII transition metal. Fresh ligand can be added at any time during the reaction in order to keep the concentration of free ligand constant. The novel transition metal-bisphosphite complexes used as catalysts can be synthesized before use, but the catalytically active complexes are generally formed in situ in the reaction medium from a catalyst precursor and the bisphosphite ligand of the invention.

As catalyst precursors, it is possible to use salts or complexes of the transition metals. Examples are rhodium carbonyls, rhodium nitrate, rhodium chloride, Rh(CO)₂(acac) (acac=acetylacetonate), rhodium acetate, rhodium octanoate and rhodium nonanoate.

The concentration of the metal in the reaction mixture is in the range from 1 ppm to 1000 ppm, preferably in the range from 5 ppm to 300 ppm.

The hydroformylation reaction carried out using the bisphosphites of the invention or the corresponding metal complexes is carried out by known methods, as described, for example, in J. Falbe, “New Syntheses with Carbon Monoxide”, Springer Verlag, Berlin, Heidelberg, N.Y., page 95 ff., (1980).

The reaction temperatures for a hydroformylation process using the bisphosphites or bisphosphosite-metal complexes of the invention as catalyst are in the range from 400° C. to 180° C., preferably from 75° C. to 140° C. The pressures under which the hydroformylation proceeds are 1-300 bar of synthesis gas, preferably 15-64 bar. The molar ratio of hydrogen to carbon monoxide (H₂/CO) in the synthesis gas is from 10/1 to 1/10, preferably from 1/1 to 2/1.

The catalyst or the ligand is homogeneously dissolved in the hydroformylation mixture comprising starting material (olefins) and products (aldehydes, alcohols, high boilers formed in the process). An additional solvent can optionally be used.

The starting materials for the hydroformylation are monoolefins or mixtures of monoolefins having from 2 to 25 carbon atoms and a terminal or internal C—C double bond. They can be linear, branched or cyclic and may also have a plurality of olefinically unsaturated groups. Examples are propene, 1-butene, c-2-butene, t-2butene, isobutene, butadiene, mixtures of C4-olefins, 1- or 2-pentene, 2-methyl-1-butene, 2-methyl-2-butene, 3-methyl-1-butene, 1-, 2- or 3-hexene, the C6-olefin mixture formed in the dimerization of propene (dipropene), 1-heptene, heptenes, 2- or 3-methyl-1-hexene, 1-octene, octenes, 2-methylheptenes, 3-methylheptenes, 5-methyl-2-heptene, 6-methyl-2-heptene, 2-ethyl-1-hexene, the isomeric C8-olefin mixture formed in the dimerization of butenes (dibutene), 1-nonene, nonenes, 2- or 3-methyloctenes, the C9-olefin mixture formed in the trimerization of propene (tripropene), decenes, 2-ethyl-1-octene, dodecenes, the C12-olefin mixture formed in the tetramerization of propene or the trimerization of butenes (tetrapropene or tributene), tetradecenes, hexadecenes, the C16-olefin mixture formed in the tetramerization of butenes (tetrabutene) and olefin mixtures prepared by cooligomerization of olefins having different numbers of carbon atoms (preferably from 2 to 4), if appropriate after fractional distillation to give fractions having the same number or a similar number of carbon atoms. It is likewise possible to use olefins or olefin mixtures produced by the Fischer-Tropsch synthesis, and also olefins obtained by oligomerization of ethene or those which can be obtained via metathesis reactions or telomerization reactions.

Preferred starting materials are propene, 1-butene, 2-butene, 1-hexene, 1-octene, dimers and trimers of butene (dibutene, di-n-butene, di-isobutene, tributene) and α-olefins in general.

The hydroformylation can be carried out continuously or batchwise. Examples of industrial apparatuses are stirred vessels, bubble columns, jet nozzle reactors, tube reactors and loop reactors, some of which may be connected in series to form a cascade and/or be provided with internals.

The reaction can be carried out in one or more stages. The separation of the aldehyde compounds formed and the catalyst can be carried out by a conventional method such as fractionation. This can, for example, be carried out industrially by distillation or by means of a falling film evaporator or a thin film evaporator. This is particularly useful when the catalyst is separated as a solution in a high-boiling solvent from the lower-boiling products. The catalyst solution which has been separated off can be used for a further hydroformylation. When using lower olefins (e.g. propene, butene, pentene), the products can also be discharged from the reactor via the gas phase.

The following examples illustrate the invention but do not restrict its scope which is defined by the claims.

### EXAMPLES

All preparations were carried out under protective gas using the standard Schlenk technique. The solvents were dried over suitable desiccants before use.

### Example 1

Synthesis of the Ligand III a

The hydroxyphosphite (CAN 108609-96-7) used as precursor was synthesized as described in U.S. Pat. No. 4,885,401.

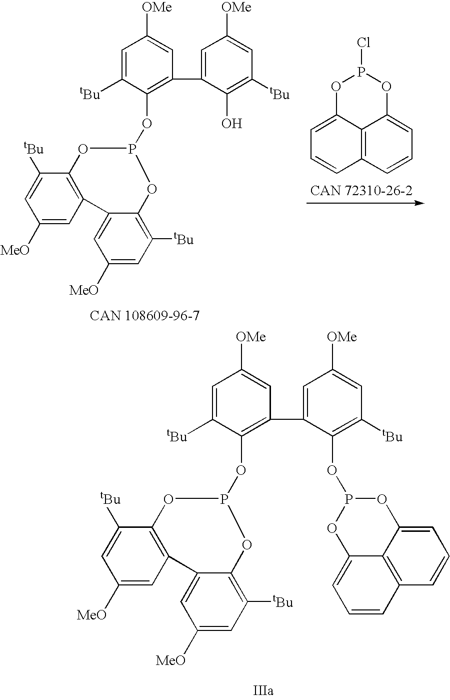

25.69 g (34.5 mmol) of the phosphite (CAN 108609-96-7) and 8.1 g of triethylamine are dissolved in 100 ml of toluene with stirring. This solution is slowly added dropwise at −40° C. to a solution of 8.0 g (35.6 mmol) of the chlorophosphite (CAN 72310-28-2) in 100 ml of toluene. After the addition is complete, the mixture is allowed to warm to room temperature and is then heated at 60° C. for 3 hours. After cooling to room temperature, the solid which has precipitated is filtered off and discarded. The solvent is removed under reduced pressure and the residue is stirred with 200 ml of pentane. The solid obtained here is isolated, washed with acetonitrile and dried under reduced pressure. Yield: 20.2 g (63%).

¹H NMR (C₇D₈) δ=7.2-6.2 (14 H, Ar—H), 3.5-3.0 (12 H, OMe), 1.4-0.9 (36 H, ^{t}Bu)

³¹P{¹H} NMR (C₇D₈) δ=134, 108 ppm

### Example 2

Synthesis of the Ligand I a

9.50 g (26.5 mmol) of 3,3′-di-t-butyl-2,2′-dihydroxy-5,5′-dimethoxybiphenyl together with 12.50 g of triethylamine are dissolved in 110 ml of toluene. 11.96 g (53.3 mmol) of the chlorophosphite component (CAN 72310-28-2) dissolved in 100 ml of toluene are added to this solution over a period of one hour. After the addition is complete, the solution is stirred for another 3 hours, the triethylammonium chloride which has precipitated is filtered off and the solution is evaporated to dryness. Recrystallization from acetonitrile gives the bisphosphite I a (49% yield).

¹H NMR (C₇D₈) δ=7.2-6.4 (16 H, Ar—H), 3.9-3.7 (6 H, OMe), 1.15 (18 H, ^{t}Bu)

³¹P NMR (C₇D₈) δ=105.9 ppm

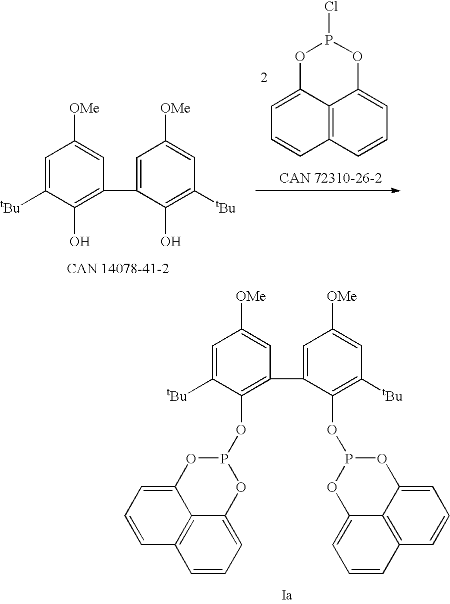

### Example 3

Hydroformylation of 1-octene

The experiments were carried out in a 300 ml laboratory autoclave (from Berghof) equipped with an internal thermometer and a capillary for taking samples during the reaction. The olefin and part of the solvent are initially placed in the autoclave, and the catalyst comprising Rh precursor and ligand dissolved in the remainder of the solvent is added from a pressure burette at the start of the reaction. In all autoclave experiments, rhodium nonanoate is used as Rh precursor.

60 g of 1-octene were hydroformylated in 100 g of toluene in an autoclave of this type. The results are summarized in the following table.

| Example No. | E3-1 | E3-2 | E3-3 | E3-4 |
|---|---|---|---|---|
| Ligand No. | 3.a | 3.a | 3.a | 3.a |
| Temperature [° C.] | 100 | 100 | 100 | 100 |
| L/Rh [mol/mol] | 5 | 4 | 5 | 8 |
| Synthesis gas pressure [bar] | 20 | 20 | 50 | 20 |
| Concentration of Rh [ppm] | 41 | 41 | 43 | 44 |
| Olefin conversion | 91.5 | 92.3 | 94.7 | 93.2 |
| Analysis of aldehydes | | | | |
| Nonanal | 81.9 | 85.1 | 81.5 | 84.4 |
| 2-Methyloctanal | 15.8 | 13.5 | 17.2 | 13.8 |
| 3-Ethylbeptanal | 2.0 | 1.3 | 1.3 | 1.5 |
| 4-Propylhexanal | 0.3 | 0.1 | 0.0 | 0.3 |

### Example 4

Hydroformylation of a Mixture of Octenes

In the autoclave described in Example 3, 60 g of a mixture of octenes (3.1% of 1-octene, 49.0% of 2-octene, 33.0% of 3-octene, 14.9% of 4-octene) dissolved in 100 g of toluene were hydroformylated. The reaction was monitored by taking samples, and the results after a reaction time of 8 hours are summarized in the following table.

| Example No. | E4-1 | E4-2 |
|---|---|---|
| Ligand No. | 3.a | 3.a |
| Temperature [° C.] | 130 | 130 |
| L/Rh [mol/mol] | 5 | 5 |
| Synthesis gas pressure [bar] | 20 | 50 |
| Concentration of Rh [ppm] | 100 | 100 |
| Olefin conversion | 97.1% | 98.0 |
| Analysis of aldehydes | | |
| Nonanal | 57.6 | 36.8 |
| 2-Methyloctanal | 27.3 | 36.2 |
| 3-Ethylheptanal | 8.0 | 13.6 |
| 4-Propylhexanal | 7.1 | 13.4 |

### Example 5

Hydroformylation of Propene

In the autoclave described in Example 3, 30 g of propene dissolved in 150 g of toluene were hydroformylated. The reaction was monitored by taking samples and the results after a reaction time of 5 hours are summarized in the following table.

| Example No. | E5-1 |
|---|---|
| Ligand No. | 3.a |
| Temperature [° C.] | 80 |
| L/Rh [mol/mol] | 2.5 |
| Synthesis gas pressure [bar] | 20 |
| Concentration of Rh [ppm] | 41 |
| Olefin conversion | 98% |
| Analysis of aldehydes | |
| Butanal | 70.5 |
| 2-Methylpropanal | 29.5 |

### Example 6

Hydroformylation of Butene

In the autoclave described in Example 3, 10 g of butene dissolved in 100 g of toluene were hydroformylated. The reaction was monitored by taking samples and the results after a reaction time of 5 hours are summarized

| Example No. | E6-1 | E6-2 | E6-3 |
|---|---|---|---|
| Ligand No. | 3.a | 3.a | 3.b |
| Olefin | 1- | t2- | c2- |
| Temperature [° C.] | butene | butene | butene |
| L/Rh [mol/mol] | 80 | 80 | 80 |
| Synthesis gas pressure | 2.5 | 2.5 | 2.5 |
| [bar] | 20 | 20 | 20 |
| Concentration of Rh [ppm] | 60 | 60 | 60 |
| Olefin conversion | 95.3% | 44.4% | 68.0% |
| Analysis of aldehydes | | | |
| Pentanal | 83.8 | 24.6 | 25.8 |
| 2-Methylbutanal | 16.2 | 75.4 | 74.2 |

1. A bisphosphite of the formula I 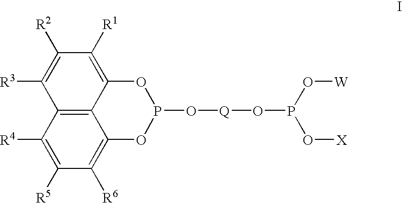
where
R¹, R², R³, R⁴, R⁵, R⁶=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR⁷, —COR⁷, —CO₂R⁷, —CO₂M, —SR⁷, —SO₂R⁷, —SOR⁷, —SO₃R⁷, —SO₃M, —SO₂NR⁷R⁸, NR⁷R⁸, N═CR⁷R⁸, NH₂, where R¹ to R⁶ may be identical or different and may be covalently linked to one another,
R⁷, R⁸=H or a substituted or unsubstituted, aliphatic or aromatic hydrocarbon radical having from 1 to 25 carbon atoms, where R⁷ and R⁸ may be identical or different,
M=an alkali metal ion, alkaline earth metal ion, ammonium ion or phosphonium ion,
Q=a divalent aliphatic, alicyclic, aliphatic-alicyclic, heterocyclic, aliphatic-heterocyclic, aromatic or aliphatic-aromatic hydrocarbon radical having from 1 to 50 carbon atoms,
W, X=aliphatic, alicyclic, aliphatic-alicyclic, heterocyclic, aliphatic-heterocyclic, aromatic or aliphatic-aromatic hydrocarbon radicals having from 1 to 50 carbon atoms, which may be identical or different or may be covalently linked to one another.

2. A bisphosphite as claimed in claim 1, characterized in that
W and X are aliphatic, alicyclic, aliphatic-alicyclic, heterocyclic, aliphatic-heterocyclic, aromatic or aliphatic-aromatic hydrocarbon radicals which have from 1 to 50 carbon atoms and are covalently linked as in the formula II 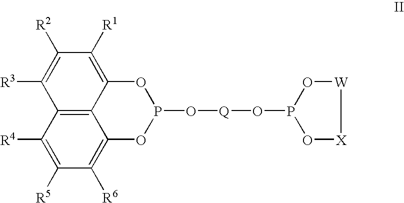
and R¹, R², R³, R⁴, R⁵, R⁶ and Q are as defined in claim 1.

3. A bisphosphite as claimed in claim 1, characterized in that
W and X are aromatic hydrocarbon radicals which have from 1 to 50 carbon atoms and are covalently linked as in the formula III 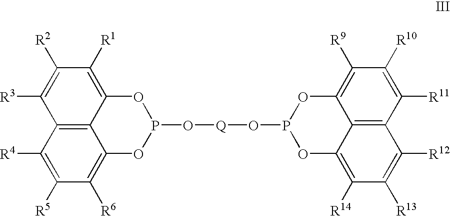
where
R⁹, R¹⁰, R¹¹, R¹², R¹³, R¹⁴=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR¹⁵, —COR¹⁵, —CO₂R¹⁵, —CO₂M, —SR¹⁵, —SO₂R¹⁵, —SOR¹⁵, —SO₃R¹⁵, —SO₃M, —So₂NR¹⁵R¹⁶, NR¹⁵R¹⁶, N═CR¹⁵R¹⁶, NH₂, where R⁹ to R¹⁴ are identical or different and may be covalently linked to one another,
R¹⁵, R¹⁶=H or a substituted or unsubstituted, aliphatic or aromatic hydrocarbon radical having from 1 to 25 carbon atoms, where R¹⁵ and R¹⁶ may be identical or different,
M =an alkali metal ion, alkaline earth metal ion, ammonium ion or phosphonium ion and
R¹, R², R³, R⁴, R⁵, R⁶ and Q are as defined in claim 1.

4. A bisphosphite as claimed in any of claims 1 to 3, characterized in that Q is a hydrocarbon radical of the formula IV 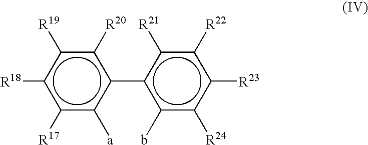
where
R¹⁷, R¹⁸, R¹⁹, R²⁰, R²¹, R²², R²³, R²⁴=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR²⁵, —COR²⁵, —CO₂R²⁵, —CO₂M, —SR²⁵, —SO₂R²⁵, —SOR²⁵, —SO₃R²⁵, —SO₃M, —SO₂NR²⁵R²⁶, NR²⁵R²⁶, N═CR²⁵R²⁶, NH₂, where R¹ to R⁶ are identical or different and may be covalently linked to one another,
R²⁵, R²⁶=H or a substituted or unsubstituted, aliphatic or aromatic hydrocarbon radical having from 1 to 25 carbon atoms,
M=an alkali metal ion, alkaline earth metal ion, ammonium ion or phosphonium ion,
where the positions a and b serve as linkage points.

5. A bisphosphite-metal complex comprising a metal of transition group 4, 5, 6, 7 or 8 of the Periodic Table of the Elements and one or more bisphosphites of the formula I 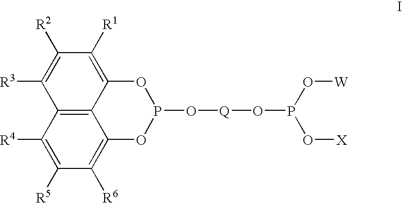
where R¹, R², R³, R⁴, R⁵, R⁶=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR⁷, —COR⁷, —CO₂R⁷, —CO₂M, —SR⁷, —SO₂R⁷, —SOR⁷, —SO₃R⁷, —SO₃M, —SO₂NR⁷R⁸, NR⁷R⁸, N═CR⁷R⁸, NH₂, where R¹ to R⁶ may be identical or different and may be covalently linked to one another,
R⁷, R⁸=H or a substituted or unsubstituted, aliphatic or aromatic hydrocarbon radical having from 1 to 25 carbon atoms, where R⁷ and R⁸ may be identical or different,
M=an alkali metal ion, alkaline earth metal ion, ammonium ion or phosphonium ion,
Q=a divalent aliphatic, alicyclic, aliphatic-alicyclic, heterocyclic, aliphatic-heterocyclic, aromatic or aliphatic-aromatic hydrocarbon radical having from 1 to 50 carbon atoms,
W, X=aliphatic, alicyclic, aliphatic-alicyclic, heterocyclic, aliphatic-heterocyclic, aromatic or aliphatic-aromatic hydrocarbon radicals having from 1 to 50 carbon atoms, which may be identical or different or may be covalently linked to one another.

6. A bisphosphite-metal complex as claimed in claim 5, characterized in that
W and X are aliphatic, alicyclic, aliphatic-alicyclic, 5 heterocyclic, aliphatic-heterocyclic, aromatic or aliphatic-aromatic hydrocarbon radicals which have from 1 to 50 carbon atoms and are covalently linked as in the formula II 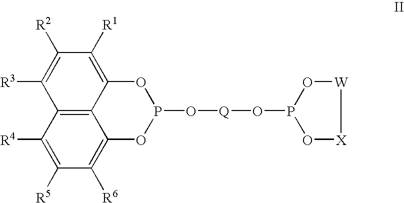
and R¹, R², R³, R⁴, R⁵, R⁶ and Q are as defined in claim 1.

7. A bisphosphite-metal complex as claimed in claim 5, characterized in that
W and X are aromatic hydrocarbon radicals which have from 1 to 50 carbon atoms and are covalently linked as in the formula III 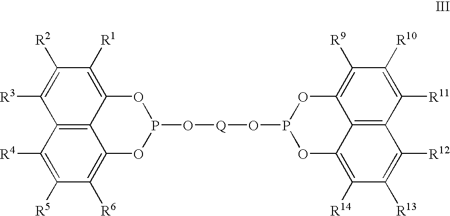
where
R⁹, R¹⁰, R¹¹, R¹², R¹³,R¹⁴=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR¹⁵, —COR¹⁵, —CO₂R¹⁵, —CO₂M, —SR¹⁵, —SO₂R¹⁵, —SOR¹⁵, —SO₃R¹⁵, —SO₃M, —SO₂NR¹⁵R¹⁶, NR¹⁵R¹⁶, N═CR¹⁵R¹⁶, NH₂, where R⁹ to R¹⁴ are identical or different and may be covalently linked to one another,
R¹⁵, R¹⁶=H or a substituted or unsubstituted, aliphatic or aromatic hydrocarbon radical having from 1 to 25 carbon atoms, where R¹⁵ and R¹⁶ may be identical or different,
M=an alkali metal ion, alkaline earth metal ion, ammonium ion or phosphonium ion and
R¹, R², R³, R⁴, R⁵, R⁶ and Q are as defined in claim 1.

8. A bisphosphite-metal complex as claimed in any of claims 5 to 7, characterized in that
Q is a hydrocarbon radical of the formula IV 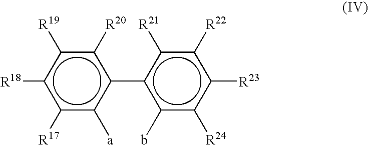
where
R¹⁷, R¹⁸, R¹⁹, R²⁰, R²¹, R²², R²³, R²⁴=H, an aliphatic, alicyclic, aliphatic-alicyclic, heterocyclic, aliphatic-heterocyclic, aromatic or aliphatic-aromatic hydrocarbon radical having from 1 to 50 carbon atoms, F, Cl, Br, I, —OR²⁵, —COR²⁵, —CO₂R²⁵, —CO₂M, —SR²⁵, —SO₂R²⁵, —SOR²⁵, —SO₃R²⁵, —SO₃M, —SO₂NR²⁵R²⁶, NR²⁵R²⁶, N═CR²⁵R²⁶, NH₂, where R¹⁷ to R²⁴ are identical or different and may be covalently linked to one another,
R²⁵, R²⁶=H or a substituted or unsubstituted, aliphatic or aromatic hydrocarbon radical having from 1 to 25 carbon atoms,
M=an alkali metal ion, alkaline earth metal ion, ammonium ion or phosphonium ion,
where the positions a and b serve as linkage points.

9. A bisphosphite-metal complex as claimed in any of claims 5 to 8, characterized in that
the metal used is rhodium, platinum, cobalt or ruthenium.

10. The use of a bisphosphite as claimed in any of claims 1 to 4 in a process for the hydroformylation of olefins.

11. The use of a bisphosphite-metal complex as claimed in any of claims 5 to 9 in a process for the hydroformylation of olefins.

12. The use of a bisphosphite as claimed in any of claims 1 to 4 in a process for the hydroformylation of olefins in the presence of further phosphorus-containing ligands.

13. The use of a bisphosphite-metal complex as claimed in any of claims 5 to 9 in a process for the hydroformylation of olefins in the presence of further phosphorus-containing ligands.
